(19) United States
(12) Patent Application Publication
(10) Pub. No.: US 2010/0288271 A1
(43) Pub. Date: Nov. 18, 2010

(54) System for Artificial Respiration of Persons

(75) Inventors: Wilhelmus Petrus Johannes De Kruijf, Enschede (NL); Klaas Jacobus Blacquiere, Deventer (NL); Evert Jan Klip, Dalen (NL); Jeroen Mathijn Wissink, Enschede (NL); Marco Voogsgerd, Enschede (NL)

Correspondence Address: THE WEBB LAW FIRM, P.C., 700 KOPPERS BUILDING, 436 SEVENTH AVENUE, PITTSBURGH, PA, 15219, US

(73) Assignee: INDES HOLDING B.V., Enschede (NL)

(21) Appl. No.: 12/514,387

(22) PCT Filed: Nov. 9, 2007
(86) PCT No.: PCT/NL07/50552

§ 371 Date: May 10, 2010

(30) Foreign Application Priority Data
Nov. 9, 2006 (NL) 2000309

(51) Int. Cl. A61M 11/00

(52) U.S. Cl. 128/200.21

(57) ABSTRACT

The invention relates to a system for artificial respiration of persons, comprising: a respirator apparatus, comprising: a respirator device and at least one respiratory tube coupled to the respirator device, which respiratory tube is adapted to enable respiration of a person; and at least one atomizing device for atomizing an additive in an airflow to be inhaled by the persons, comprising: at least one atomized body coupled to the respiratory tube and at least one supply container for holding a supply of at least one additive.

The invention relates to a system for artificial respiration of persons.

It is known that, while artificial respiration is being applied to a person, it is also possible to administer one or more medications to the person in question. Administering of the medication to the person can herein take place in pulmonary manner, wherein the medication is introduced into the person via the lungs. The human lungs generally comprise about 3 million alveoli with a total surface area of more than 200 m². Due to the relatively large contact surface of the lungs, the lungs are in general particularly suitable for administering medications to the body of the person in relatively simple, efficient and immediate manner. The lungs are generally suitable for effective absorption of both low-molecular and high-molecular medications. In order to enable a medication to be administered to a person, a medication will generally have to be converted into an aerosol, a fine powdery mist or a form of vapour for an adequate assimilation in the respiratory tract and/or the lungs. In order to realize a desired control of the dosage, penetration depth and deposition speed of the dispersed medication in the respiratory tract and/or the lungs, special inhaler devices are generally applied.

A system for artificial respiration of persons and simultaneous pulmonary administering of a medication via the airflow to be inhaled is known from American patent US 2004/0084050. This patent particularly describes a method and a system for administering nebulized medicines to persons by making use of a catheter, which catheter will be partially introduced into a person for pulmonary treatment. A proximal end of the catheter which will be introduced into the person for treating is provided with a nebulizing body for nebulizing a medicine to small droplets. An opposite distal end of the nebulizing element is connected to a supply container for the medicine. Since a relatively short catheter with a length of a maximum of 45 centimetres is applied, the supply container will be positioned in the vicinity of the patient. The person for treating is optionally also artificially respirated to using a respiratory tube which can be arranged partially in the patient. The catheter and the respiratory tube can herein be mutually connected and optionally mutually integrated, wherein the medicine can be nebulized in an airflow to be inhaled. A control unit can be applied to synchronize the nebulizing process and the artificial respiration of the person. Specifically described in this patent is an air-driven nebulizing body (also frequently referred to as air-assisted atomizer), wherein the medication can be nebulized using pressurized air. A porous material manufactured by Porex can optionally be applied as nebulizing element through which the medication and the pressurized air are guided. Although it is particularly advantageous to combine, and preferably integrate, the pulmonary administering of a nebulized medication with the artificial respiration of a person, this embodiment of the known system has substantial drawbacks. A significant drawback of this embodiment of the known system is that the nebulizing element must be positioned on the proximal end of the catheter and of the respiratory tube, and so always inside the person, in order to allow relatively efficient pulmonary administration of the medication. The reason for this is that using the known system only a relatively broad droplet size distribution can be realized with a relatively large geometric standard deviation (GSD) lying between 2 and 2.5. If the nebulizing element were to be connected at a distance from the patient to the respiratory tube, a large part of the nebulized medication would condense on an inner wall of the respiratory tube and the person would actually only be able to inhale a small fraction. Because the relatively bulky assembly of the respiratory tube and the catheter provided with the nebulizing element must be arranged in the person, this will have a substantial adverse effect on the comfort of the person. Furthermore, the introduction of this voluminous assembly will increase the risk of (additional) inflammations or infections, which will also be detrimental to the interests of the person to be treated. In addition, the assembly of the respiratory tube and the catheter is structurally relatively complex, which is generally unfavourable from an economic and logistic viewpoint. It is also often the case that when the respiratory tube is introduced it is not yet known whether a medicine will have to be administered at a later stage to the person to be respirated. Standard application of the known construction is also disadvantageous from an economic viewpoint. It is generally too expensive to provide as standard every person to be respirated with a catheter, since administering of a medication is not always necessary. An additional drawback of the known system is that the supply container is situated close to the patient, and therefore very much in the sterile zone of the patient who is generally protected (by drapes), and this is unfavourable because an anesthetist must gain access to the protected sterile zone in order to be able to see whether the supply container is empty and must be replaced, which further increases the chance of for instance infections in the person to be treated.

The invention has for its object to provide an improved system for artificial respiration of persons, wherein at least one additive can be simultaneously administered in relatively user-friendly manner.

The invention provides for this purpose a system of the type stated in the preamble, comprising: a respirator apparatus, comprising: a respirator device and at least one respiratory tube coupled to the respirator device, which respiratory tube is adapted to enable respiration of a person; and at least one atomizing device for atomizing an additive in an airflow to be inhaled by the person, comprising: at least one substantially plate-like, passive atomizer body provided with at least one perforation defining an atomizing channel, at least one supply holder for holding a supply of at least one substantially liquid additive, at least one administering conduit for the additive connected to the supply container and the at least one atomizing channel of the atomizer body, and pressure-generating means coupled to the supply container to enable a pressure to be exerted on the additive such that the additive can be atomized by means of the atomizer body into the airflow to be inhaled, wherein the at least one atomizing channel of the passive atomizer body has a diameter of a maximum of 5 micrometres, and wherein the length of the administering conduit amounts to at least one metre. By applying a passive and substantially plate-like atomizer body (usually also referred to as plain orifice atomizer) provided with at least one perforation defining an atomizing channel and having a diameter of a maximum of 5 micrometres, the additive can be atomized into small droplets of about 10 micrometres, wherein the droplet size distribution is relatively limited, in particular to a droplet size distribution with a relatively favourable geometric standard deviation of between 1.04 and 1.6. The combination of the relatively small droplet size and the relatively limited droplet size distribution has the result that the atomized additive will substantially not deposit against an inner wall of the respiratory tube and that it will thus be possible for substantially all the atomized additive to the absorbed by the person for respirating, this making it possible to allow the atomization of the additive to take place in relatively efficient manner outside (at a distance from) the person. In the case a larger droplet size and/or a broader droplet size distribution were to be realized through the perforated atomizer body, it will then not be possible for pulmonary administering of the person to be respirated to take place in efficient manner because a substantial part of the atomized additive will condense in the respirator apparatus, and usually on an inner wall of the respiratory tube. Because the system according to the invention is adapted to enable efficient atomizing of the additive at a distance from the person to be respirated, the additive can be administered in relatively comfortable manner to the person to be respirated. Furthermore, only a part of the respiratory tube (so not the administering conduit provided with the respirator body) will in this way have to be arranged in the person, which considerably reduces the chance of infections in the person. In addition, a relatively inexpensive respiratory tube can be arranged in the person, to which tube the respirator body can be connected at a later stage in optionally releasable manner. The passive, substantially plate-like perforated atomizer body (also referred to as plain orifice atomizer) is characterized in that this atomizer body is adapted to atomize a substantially liquid, in particular aqueous additive, by pressing the additive at a pressure of at least 1.5 bar through the at least one atomizing channel, whereby so much turbulence and so many friction forces are developed in the liquid jet generated by the atomizer body that the jet will break apart into a finally distributed mist with a limited droplet size distribution. No separate atomizing medium, such as a gas, is thus required for the purpose of atomizing the additive. The atomizing channel thus functions in fact as exit channel for the additive from the administering conduit which can be atomized via the atomizing channel and can be administered to the airflow to be inhaled. Because a passive atomizer body is applied and not an active atomizer body, such as for instance a piezoelectric atomizer body, an additional advantage is that the administering conduit used can be relatively long, wherein the length of the administering conduit in the system according to the invention amounts to a minimum of one metre, whereby the supply container for the additive can be placed at a distance from the person to be treated and outside the sterile zone, this being advantageous from a practical viewpoint for both the person to be respirated and the anesthetist, and from the viewpoint of sterility, whereby the occurrence of inflammations in the person to be respirated can further be avoided. The person to be respirated therefore no longer need be burdened with the supply container and an anesthetist can in this way gain access to the supply container in relatively simple manner. In addition to positioning the supply container at a distance from the person to be respirated, the pressure-generating means co-acting with the supply container will generally also be positioned at a distance from the person. Because both the supply container and the pressure-generating means can be positioned at a distance from the person to be respirated, the design freedom in respect of these two components of the atomizing device can be increased considerably, whereby the atomizing device can be optimized for specific applications in relatively simple and efficient manner. The additive for administering to a person (or animal) by making use of the system according to the invention can be of very diverse nature. The additive will generally comprise at least one medication. In addition, it is also possible to envisage administering non-pharmaceutical medications, such as for instance water, to a person to be respirated. It is noted that artificial respiration of persons is also understood to mean the (artificial) support of the breathing of persons. It is also noted that the system will generally only be applied for the respiration of persons. It is however also possible to envisage the system according to the invention being used for artificial respiration of animals, wherein one or more additives can be pulmonarily administered simultaneously to a determined animal.

Although the system according to the invention could also be applied for atomizing the additive inside the person, it is however recommended to have the atomization take place outside the person as stated in the foregoing. It is therefore advantageous for the atomizer body to be coupled to the respirator apparatus such that atomizing of the additive in the airflow takes place at a distance from the person to be respirated. The atomizer body is preferably coupled to a half of the respiratory tube directed toward the person. It is possible in this way to realize that a maximum quantity of atomized additive will actually be inhaled by the person, this enhancing the efficiency of the pulmonary administration using the system. In another preferred embodiment the atomizer body is coupled to the respirator apparatus such that atomizing of the additive in the airflow takes place at a distance of a maximum of 20 centimetres from the person to be respirated, in order to further increase the efficiency of the system according to the invention.

The proximal end (located close to the person) of the respiratory tube can be of very diverse nature. In a preferred embodiment the proximal outer end of the respiratory tube is adapted to be at least partially inserted into the person to be respirated. The proximal outer end can be provided for this purpose with an intubation element such as an intubation tube, which can be inserted at least partially into the person via for instance the mouth, the nose, or via a tracheal incision. This embodiment is generally recommended for medical reasons in the respiration of persons who are unconscious. In order to enable optimization of the effectiveness of the pulmonary administration the length of the intubation element is preferably such that the intubation element can be inserted beyond the vocal chords into the person. The atomizer body is preferably adapted to atomize the additive in the airflow at a distance from the part of the intubation element inserted into the person to be respirated, thus outside the person. In an alternative preferred embodiment the proximal outer end of the respiratory tube can be provided with an optionally flexible respiratory cover (respiratory mask) which encloses the mouth and/or the nose of the person to be respirated. In a particular preferred embodiment the atomizer body is coupled to the intubation element and/or the respiratory cover. In this manner the additive in the respective device can be atomized very close to the person, which will generally further enhance the effectiveness of the pulmonary administration. It is noted that it is not necessary for the additive to be atomized in the respiratory tube, but that the additive can also be atomized close to (at a distance from) the respiratory tube such as for instance in a respiratory cover. The atomizer body is however preferably connected to a part of the respiratory tube located a distance from the intubation element, to a part of the intubation element not to be inserted (or already inserted), into the person and/or to the respiratory cover, in order to enable atomizing of the additive at a distance from the person. Although the additive can also be atomized at a distance from the respiratory conduit, it is however recommended to atomize the additive in an airflow to be inhaled by the person, whereby the additive can be inhaled in relatively efficient manner and can thus be administered pulmonarily in relatively efficient manner.

As already noted above, the structural construction of the atomizer body, among other factors, is important in realizing a limited droplet size and droplet size distribution such that the system according to the invention is (also) adapted to atomize the additive at a distance from a person to be respirated. It is also advantageous here when the diameter is substantially constant in the length direction of the at least one atomizing channel, and thus amounts to a maximum of 5 micrometre. The atomizing channel will moreover take a substantially linear form. In this way the droplet size (distribution) to be realized can already be predefined relatively accurately.

The atomizer body generally comprises a plurality of perforations, wherein each perforation defines an atomizing channel, and wherein each atomizing channel (or each perforation) has a more preferably substantially identical diameter of a maximum of 5 micrometres. The perforations are arranged at predetermined locations in the atomizer body. The number of atomizing channels depends on the quantity of additive to be dosed per inhalation and can vary from two to several tens, hundreds or even thousands of atomizing channels. The mutual distance between the atomizing channels amounts in a preferred embodiment to a maximum of 50 micrometres. The atomizing channels can in this way be arranged relatively efficiently and compactly, although the atomizing channels are positioned a sufficient distance from each other to be able to guarantee optimum atomizing of the additive. The atomizing channels are preferably in a regular mutual arrangement, and more preferably arranged in line and still more preferably such that the atomizing channels are arranged mutually in line and substantially transversely of the airflow to be inhaled. An efficient absorption of the atomized additive by the airflow to be inhaled can in this way be realized, whereby an aerosol can be formed which can be inhaled optimally.

The droplet size of the additive atomized by the atomizer body must be sufficiently small, and preferably smaller than 10 micrometres, in order to enable optimizing of the pulmonary administration of the additive. It is therefore advantageous when the atomizer body is adapted to generate an atomized additive with an average droplet size of a maximum of 10 micrometres. The droplet size will generally be substantially equal to twice the diameter of the atomizing channel in the case a minimal operating pressure of 1.5 bar is employed to enable the realization of a so-called Rayleigh break-up of a liquid jet (additive jet) from the atomizer body. The droplet size can be regulated by adjusting the diameter of the atomizing channel. The atomizer body is preferably adapted to generate an atomized additive with a geometric standard deviation (GSD) lying between 1.001 and 1.2. Such an advantageous GSD can be achieved with the passive, substantially plate-like atomizer body provided with one or more atomizing channels with a maximum diameter of 5 micrometres. It is noted that a GSD of 1.2 or less is usually also designated a monodisperse droplet distribution. In the case the average droplet size amounts to 5 micrometre and the GSD amounts to 1.2, 80% of the droplets will then have a droplet size lying between 4 and 6 micrometres.

In a preferred embodiment the length of the at least one atomizing channel amounts to a maximum of 02 millimetre. A channel length of a maximum of 0.2 millimetre has the advantage that the operating pressure of the system can be kept lower than 50 bar. At pressures higher than 50 bar problems will usually occur in realizing sufficient sealing of the system so as to prevent leakage from the system. The pressure-generating means are preferably adapted to exert on the additive an operating pressure p substantially equal to

$$p = \frac{32 \cdot v \cdot η \cdot L}{{D}^{2}} ,$$

wherein < relates to the velocity of the additive, 0 to the viscosity of the additive, L to the length of the atomizing channel and D to the diameter of the atomizing channel. The velocity < of the additive generally amounts to 20 m/s, the viscosity 0 of the additive amounts to 1.0020 mPaθs and the diameter D of the atomizing channel to a maximum of 5 micrometres. In the case a maximum operating pressure of 50 bar (50θ10⁵ Pa) is allowed, it then follows from the above formula (a derivation of Poiseuille's law) that the channel length may amount to a maximum of 0.2 millimetres. An operating pressure of between 5 and 25 bar will usually be applied which, according to the above formula, affects the peripheral condition(s) for the diameter D or the length L of the atomizing channel. The pressure-generating means must herein be adapted to exert on the additive an operating pressure of a maximum of 50 bar, in particular an operating pressure lying between 5 and 25 bar.

The atomizer body is preferably manufactured at least partially from at least one of the following materials: plastic, metal and ceramic. Silicon is generally recommended as ceramic material since silicon is relatively inexpensive and relatively easy to process to a durable (small-scale) canalized structure. In a preferred embodiment the administering conduit and the atomizer body take a disposable form. It is also possible to envisage the supply container and/or possible other components of the atomizing device taking a disposable form. A disposable form of at least a part of the atomizing device is usually particularly advantageous from a hygienic viewpoint, because the chance of contamination of the atomizing device after use, and thereby contamination and in particular infection of the person to be respirated, can be prevented.

Although the diameter of the at least one atomizing channel may amount to a maximum of 5 micrometres, the diameter of the at least one atomizing channel more preferably lies substantially between 0.5 and 5 micrometres. A channel diameter smaller than 0.5 micrometre will generally result in too small a droplet size to be able to result in an efficient pulmonary administration of the additive.

In order to enable positioning of the supply container (and the pressure-generating means) at a distance from the person to be respirated, and more preferably outside the sterile zone—if present—around the person to be respirated, the length of the administering conduit must, as indicated, be sufficiently great and amount to a minimum of 1 metre. The administering conduit is preferably at least 150 cm and in particular at least 250 cm. A greater length of the administering conduit will generally improve the freedom in positioning the supply container. From a practical viewpoint the maximum length of the administering tube will generally amount to a maximum of 300 cm. At a relatively great length (above 300 cm) of the administering conduit the pressure drop over the administering conduit will moreover also be relatively great, and this is undesirable.

While the system according to the invention is in operation the administering tube will generally be substantially fully filled with additive so that the pressure-generating means can bring about an atomization of the additive in relatively instantaneous manner. By exerting pressure on (substantially liquid) additive situated in the supply container, a pressure will also be exerted on additive present in the administering conduit, whereby a metered quantity of additive can be atomized and thereby administered in relatively accurate and instantaneous manner. In order to minimize the quantity of additive present in the administering conduit, the smallest internal diameter of the administering conduit preferably lies substantially between 0.05 and 2 mm. More preferably the internal diameter of the administering conduit is substantially constant along the length of the administering conduit. In a particular preferred embodiment the internal diameter of the administering conduit amounts substantially to 0.5 mm.

In the case a plurality of additives have to be administered to the person to be respirated, it is advantageous if the atomizing device comprises a plurality of supply containers. Each supply container can then be provided with a determined additive. It is however generally desirable in that case that each supply container is coupled to its own administering conduit so as to be able to prevent possible undesirable mixing of the additives before they are atomized. The various administering conduits can be mutually connected (and thereby form for instance a multi-lumen tube) and can be coupled to one (collective) atomizer body. It is however also possible to envisage the atomizing device comprising a plurality of atomizer bodies. The administering conduit preferably connects here to the plurality of atomizer bodies which are more preferably arranged adjacently of each other. However, in the case a plurality of administering conduits are applied it is also possible to envisage each administering conduit being coupled to its own atomizing element. The application of a plurality of supply containers can also be advantageous in the case a single additive is distributed among different supply containers, since in this manner the preserving and optional dispensing of the additive can generally be improved.

The administering conduit for the additive can be of relatively rigid nature and can for instance be formed by a rigid pipe manufactured from plastic and/or metal. It is also possible to envisage the administering conduit being formed by a (slightly) flexible tube, which will generally enhance freedom of positioning of the supply container. The administering conduit is preferably manufactured at least partly from a material substantially having bending stiffness. The administering conduit has bending stiffness here such that bending and hysteresis losses can be prevented, or at least be countered. It is noted that an administering conduit with at least partial bending stiffness does not necessarily have to be rigid, and can (to a certain extent) also be flexible.

Coupling of the atomizer body to the respiratory tube preferably takes place by making use of a separate coupling element, which coupling element is adapted for preferably releasable coupling to the respiratory tube. The coupling element can herein be formed by a sleeve which can be pushed partly over or partly into the respiratory tube. In an alternative embodiment it is possible to envisage the coupling element forming part of the respiratory tube for facilitating coupling of the atomizer body to the respiratory tube. The coupling element preferably takes a substantially T-shaped form, wherein the additive can be atomized into the airflow substantially perpendicularly of the flow direction of the airflow. The coupling element can herein taper convergently and/or divergently in the flow direction of the airflow. An inner side of the coupling element preferably defines a mixing zone for mixing the airflow (to be inhaled) with the atomized additive, wherein the area A of a cross-section of the mixing zone changes in the flow direction x of the airflow such that the change dA(x)dx at any location x in the mixing zone lies between −c₁√A(x) and 0 or between c₂√A(x) and 0, wherein c₁=15.35 or 4.22 and c₂=1.58 or 0.88 or 0.31. This guarantees that, even in the case the cross-section of the mixing zone varies in the flow direction of the airflow, the airflow does not release from the inner wall of the coupling piece, whereby turbulence in the airflow can be prevented. In the case c₂ is less than 1.53, and in particular less than 0.31, there is then the danger of the airflow releasing from the inner wall of the coupling piece, and this would result in undesirable turbulence. By causing a substantially laminar flow of the airflow the additive droplets can be held at a distance from each other and at a distance from the inner wall of the coupling piece, and the inner wall of the respiratory conduit, which further enhances the efficiency of the system according to the invention.

In a preferred embodiment the atomizing device comprises at least one closing valve for regulating passage of the additive through the administering conduit. The closing valve is herein adapted to at least partially open and close the administering conduit to respectively allow and prevent atomizing of the additive. The closing valve can for instance be formed by a tap and/or a non-return valve. If the case the administering conduit has a sufficiently flexible form, the closing valve can also be formed by a clamp engaging on the administering conduit. In a particular preferred embodiment the system comprises at least one control unit for controlling the at least one closing valve subject to the situation of the respirator device. This is because it will in general be advantageous to atomize the additive in an airflow for inhaling and it will be disadvantageous to atomize the additive in an exhaled airflow. By linking the control of the closing valve to the situation of the respirator device the additive can be administered in effective and efficient manner, wherein the exact dosage of the additive to be effectively administered can be calculated, whereby wastage of the additive can be minimized. Detection of the situation of the respirator device is preferably realized by applying one or more flow sensors in the respiratory tube, wherein the control unit is coupled to the at least one flow sensor for the purpose of controlling the at least one closing valve. The (artificial) respiration process can be monitored relatively precisely by means of the flow sensor, whereby at determined moments the additive can be atomized in an airflow to be inhaled in relatively precise manner. In yet another embodiment the respirator device can be equipped such that it generates a control signal for controlling the closing valve.

The atomizer body connecting to the administering conduit is preferably formed by a passive atomizer body. In contrast to for instance a piezo-electric element, the passive atomizer body is a non-manipulable, and therefore inert, element. The atomizer body preferably comprises at least one atomizing channel, which atomizing channel can also be deemed to be an atomizing opening. By pressing the additive through the atomizing opening the additive will be atomized to an aerosol. A passive atomizing element preferably operates in accordance with the Rayleigh spray principle (see for instance U.S. Pat. No. 6,189,813). In order to enable an increase in the capacity of the atomizer body, the atomizer body preferably comprises a plurality of atomizing channels (or atomizing openings). The plurality of atomizing channels are more preferably positioned substantially mutually in line. The atomizer body is preferably positioned in the system according to the invention such that the assembly of the atomizing channels lying in line is oriented substantially perpendicularly (transversely) relative to a passing airflow. In this manner it is possible to prevent, or at least counter, collision between the formed droplets, and thereby assimilation of a plurality of small droplets into one or more larger droplets, whereby the droplet size (distribution) of the atomized additive can be pre-determined relatively accurately. The droplet size of the additive generally determines, usually in combination with the airflow speed, how far the additive can penetrate into the airways of the person to be respirated; droplets smaller than 4 μm can potentially penetrate into the alveoli, while larger droplets will not usually reach any further than the trachea (windpipe) and the bronchi. The droplet size of the aerosol administered by the invention amounts to between 0.1 and 100 micrometres more preferably between 0.2 and 40 micrometres, and in particular between 1 and 8 micrometres.

In an alternative preferred embodiment the atomizing device and the respirator apparatus are at least partly integrated with each other. The at least partial integration of the respirator apparatus and the atomizing device will generally enable considerable simplification of the structural construction of the system according to the invention and possibly enable the volume taken up by the system to be considerably limited. An example of a possible integration of the respirator apparatus and the atomizing device that can be envisaged is the respiratory tube and the administering conduit (formed as a tube) being mutually integrated. Both tubes can here for instance form part of a multi-lumen tube. It is also possible to envisage the administering conduit being incorporated in the respiratory tube. Advantages and embodiment variants of the atomizing device have already been described at length in the foregoing.

The invention also relates to an atomizing device for use in a system according to the invention.

The invention further relates to an assembly of an administering conduit and an atomizer body for use in an atomizing device according to the invention. The assembly preferably takes a disposable form.

The invention also relates to an atomizer body for use in an assembly according to the invention.

The invention will be elucidated on the basis of non-limitative exemplary embodiments shown in the following figures. Herein:

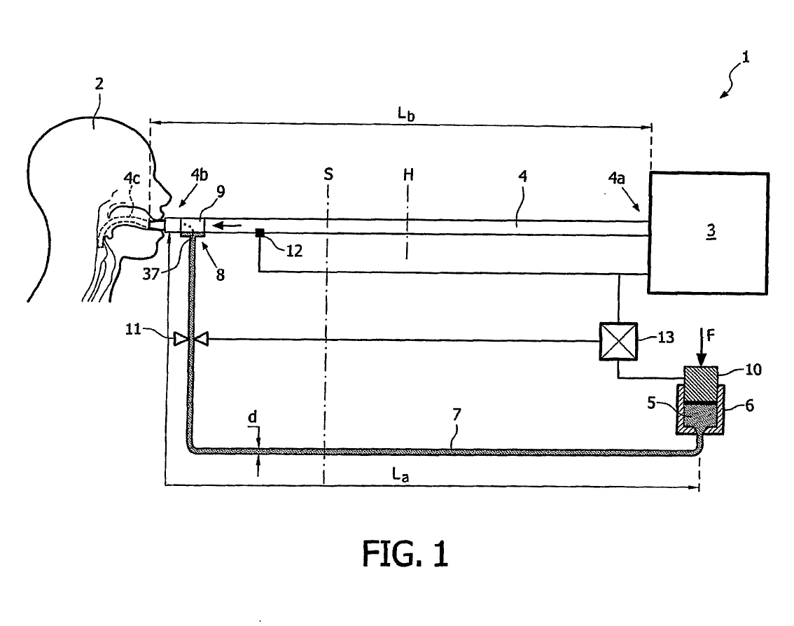

FIG. 1 shows a schematic representation of a first embodiment of a system for the artificial respiration of a patient according to the invention,

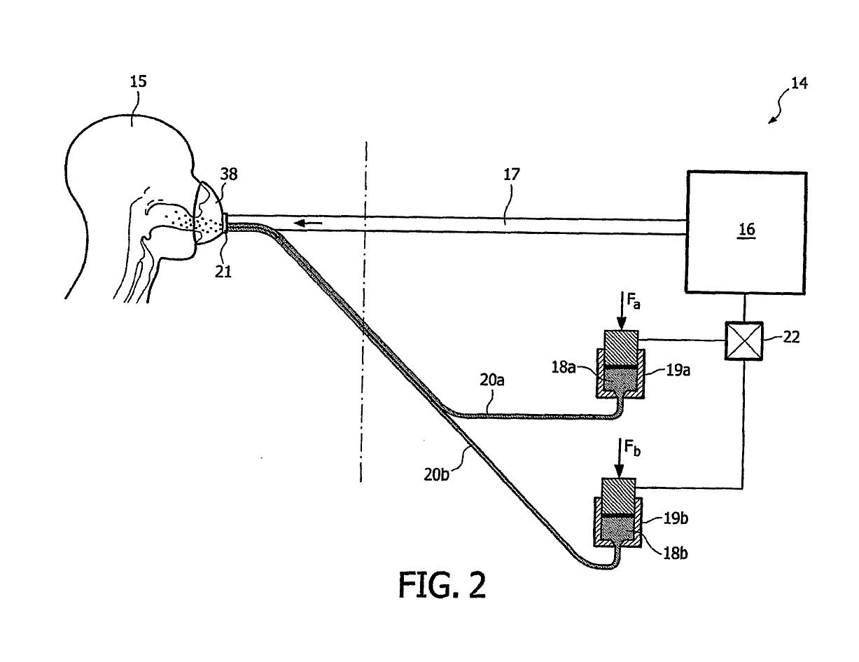

FIG. 2 shows a schematic representation of a second embodiment of a system for the artificial respiration of a patient according to the invention,

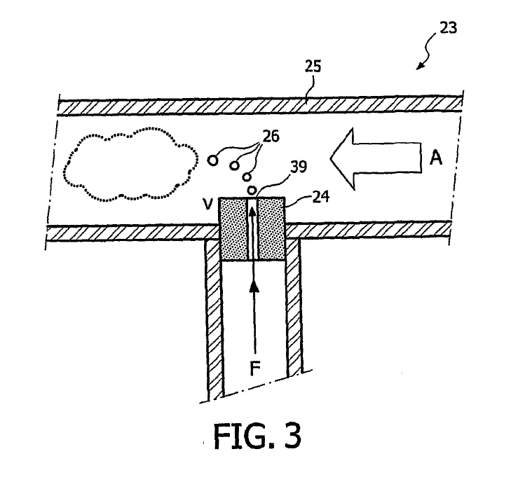

FIG. 3 shows a cross-section of an assembly of an atomizer body and a respiratory tube coupled to the atomizer body, and

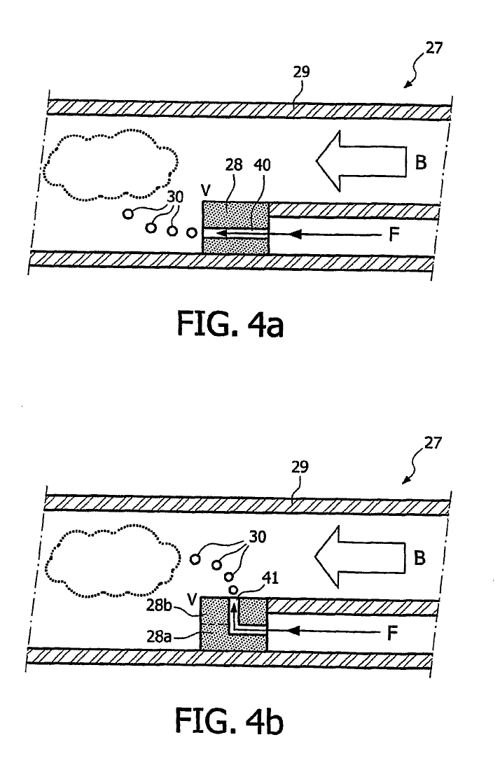

FIGS. 4a and 4b show cross-sections of another assembly of an atomizer body and a respiratory tube coupled to the atomizer body for use in a system according to the invention,

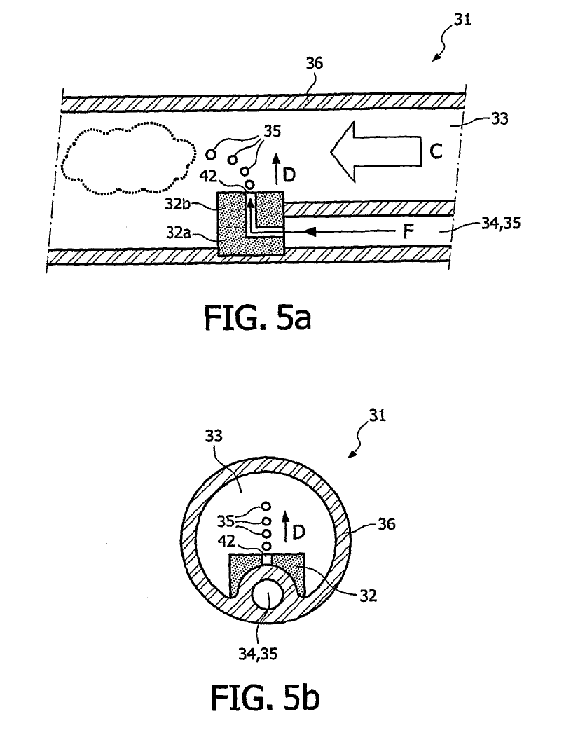

FIGS. 5a and b show cross-sections of an intubation element wherein the administering tube is integrated into the wall of the intubation element, and

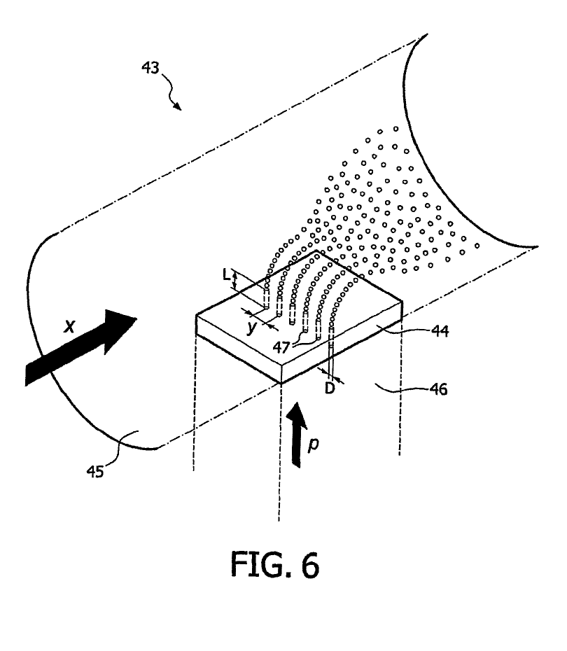

FIG. 6 shows a perspective view of a part of another system according to the invention, and

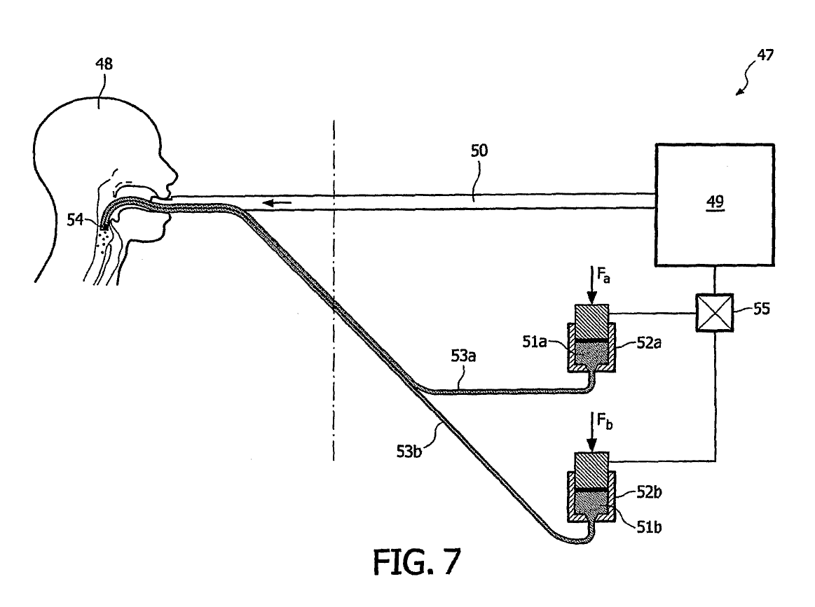

FIG. 7 shows a schematic representation of an alternative embodiment of a system for the artificial respiration of a patient according to the invention.

FIG. 1 shows a schematic representation of a first embodiment of a system 1 for artificial respiration of a patient 2 according to the invention. System 1 comprises a respirator device 3 and a respiratory tube 4 coupled to respirator device 3, wherein a distal outer end 4a of respiratory tube 4 is in fact coupled to respirator device 3 and wherein a proximal outer end 4b of respiratory tube 4 forms a mouthpiece for patient 2. An incubation tube 4c (shown with broken lines) can also be applied instead of mouthpiece 4b. As shown in FIG. 1, mouthpiece 4b is partially arranged in the patient. By activating respirator device 3 the patient 2 can be artificially respirated via respiratory tube 4 and/or the natural respiration of the patient can be (otherwise) supported. In addition to respirating the patient 2, system 1 according to FIG. 1 is also adapted for pulmonary administration of a substantially liquid medication 5. System 1 comprises for this purpose a supply container 6 for the medication 5, an administering tube 7 connected to supply container 6, which administering tube 7 is filled substantially wholly with the medication 5 and connects to a passive, substantially plate-like atomizer body 8 to enable atomizing of medication 5. Atomizer body 8 is coupled to respiratory tube 4 by means of a coupling sleeve 9 close to patient 2. FIG. 1 shows that atomizer body 8 is coupled to a half of the respiratory tube 4 directed toward the patient. For the purpose of elucidation the half of respiratory tube 4 is indicated by dividing line H. Atomizer body 8 is provided with an atomizing channel 37 for passage of medication 5, wherein the atomizing channel has a diameter of 5 micrometres and a length of 0.2 millimetre. Supply container 6 is adapted to co-act with a piston 10 to enable a force F to be exerted on medication 5, whereby medication 5 can be atomized in respiratory tube 4 and then be pulmonarily administered to patient 2. Piston 9 can for instance be driven pneumatically, hydraulically or in other manner. Instead of a piston 9 it is also possible to envisage exerting pressure on the meniscus of medication 5 by means of a gas, for instance air. In this exemplary embodiment piston 10 exerts a pressure of 50 bar on medication 5. Medication 5 can be wholly liquid but can also be partially liquid, wherein the medication 5 (which can be pumped) can for instance be formed by a (nano)suspension. Through use of administering tube 7 the supply container 6 and piston 9 co-acting therewith can be placed at a distance from patient 2, which is particularly advantageous for both patient 2 and care staff (not shown). In the shown exemplary embodiment an imaginary dividing line S is shown separating a sterile zone around patient 2 from a non-sterile zone at a distance from the patient, wherein it is apparent that both respirator device 3 and supply container 6 and piston 9 co-acting therewith are placed in the non-sterile zone. In the present exemplary embodiment the length Lₐ of administering tube 7 amount to at least 50 cm, and the length L_{b} of respiratory tube 4 amounts to about 1.5 m. In this exemplary embodiment the inner diameter d of administering tube 7 amounts to 0.5 mm, whereby the quantity of medication 5 present in administering tube 7 can be limited to a minimum. System 1 further comprises a closing valve 11 enabling selective closing of administering tube 7 for the purpose of being able to allow or block the transport of medication 5 through administering tube 7. Closing valve 11 can be positioned close to patient 2 or close to supply container 6. It is however also possible to envisage closing valve 11 being omitted, and thus not being applied. System 1 also comprises a pressure sensor 12 for monitoring (the momentary position in) the breathing process on the basis of pressure change. In order to enable medication 5 to be atomized in administering tube 4 only at selective moments during the breathing process, in particular during a (first) part of inhalation, system 1 comprises a control unit 13. In this exemplary embodiment control unit 13 is coupled to pressure sensor 12, closing valve 11 and piston 10. Piston 10, and optionally closing valve 11, will be controlled on the basis of the pressure recorded by pressure sensor 12, whereby medication 5 can be atomized in respiratory tube 4 at relatively precisely determined moments in order to enable optimization of the pulmonary administration of medication 5 and avoid wastage of medication 5.

FIG. 2 shows a schematic representation of a second embodiment of a system 14 for artificial respiration of a patient 15 according to the invention. System 14 comprises a respirator device 16 and a respiratory tube 17 coupled to respirator device 16, which respiratory tube 17 is optionally coupled releasably to a respiratory mask 38 arranged on patient 15. System 14 is also adapted to administer a plurality of medications 18a, 18b to patient 15. Medications 18a, 18b are preserved separately in a plurality of supply containers 19a, 19b, which supply containers 19a, 19b are each coupled via an administering tube 20a, 20b to an atomizer body 21 connected to respiratory mask 38 for the purpose of being able to atomize medications 18a, 18b in an airflow to be inhaled. Atomizer body 21 is provided with a plurality of atomizing channels (not further shown) for passage of medications 18a, 18b, wherein the diameter of each atomizing channel amounts to a maximum of 4 micrometres and the length of each atomizing channel amounts to 0.1 millimetre. In order to enable atomizing of medications 18a, 18b, a force Fₐ, F_{b} can be exerted selectively by pressure-generating elements (not shown) on medication 18a, 18b present in supply containers 19a, 19b, such as for instance an operating pressure of 10 bar, 15 bar respectively. These operating pressures are sufficiently high to enable reliable atomizing of the medication while being sufficiently low to enable a good sealing of system 14, whereby leakages in system 14 can be prevented. In the shown exemplary embodiment the pressure-generating elements and respirator device 16 are coupled to a control unit 22 for the purpose of enabling selective atomizing of at least one medication 18a, 18b subject to the situation of respirator device 16. Control unit 22, and optionally also supply containers 19a, 19b, can optionally be arranged integrally in respirator device 16. Administering tubes 20a, 20b are connected to each other as a multi-lumen tube and are together guided in respiratory tube 17 close to patient 15. Because the part of respiratory tube 17 situated in patient 15 encloses administering tubes 20a, 20b, the assembly of tubes 17, 20a, 20b can be arranged relatively easily and reliably in patient 15. FIG. 3 shows a cross-section of an assembly 23 of an atomizer body 24 and a respiratory tube 25 coupled to atomizer body 24. Atomizer body 24 is provided with an atomizing channel 39 through which a fluid can be guided. Atomizing channel 39 has a diameter of 3 micrometres and a length of 0.05 millimetre. An airflow to be inhaled by a person is visualized by means of arrow A. FIG. 3 clearly shows that the initial atomization of the fluid into droplets 26 takes place in a direction substantially perpendicular to the direction of flow of the airflow.

FIG. 4a shows a cross-section of another assembly 27 of an atomizer body 28 and a respiratory tube 29 coupled to atomizer body 28 for use in a system according to the invention. FIG. 4a in particular shows that direction of flow B of an airflow for inhaling is substantially parallel to the initial direction of atomization of an additive 30 to be added to the airflow. Atomization of additive 30 takes place by exerting a force F on a supply of the additive 30 in contact with atomizing element 28, wherein the force F is exerted in the direction of atomizer body 30. Atomizer body 30 is herein provided with a plurality of successively positioned atomizing channels 40, wherein each atomizing channel 40 has an internal diameter of 4.5 micrometres and a length of 0.15 millimetre.

FIG. 4b shows a cross-section the same as FIG. 4a with the outflow opening of atomizer body 28 perpendicular to airflow B. Atomizer body 28 is herein provided with an angled atomizing channel 41 which is in fact formed by laminated assembly of atomizer body 28 from a first layer 28a and a second layer 28b arranged on first layer 28a.

FIG. 5a shows a longitudinal section of yet another assembly 31 of an atomizer body 32 and a respiratory tube 33 coupled to atomizer body 32 for use in a system according to the invention. Atomizer body 32 is constructed in modular manner from a lower layer 32a and an upper layer 32b arranged on lower layer 32a in order to enable an angled form of an atomizing channel 42 arranged in atomizer body 32. FIG. 5a further shows that an administering conduit 34 for an additive 35 to be atomized in the airflow via atomizer body 32 lies substantially parallel to the direction of flow C of the airflow. In this exemplary embodiment the administering conduit 34 is arranged integrally in a wall 36 of respiratory tube 33, this also being clearly shown in the cross-section of assembly 31 shown in FIG. 5b. As shown in both FIG. 5a and FIG. 5b, the direction in which additive 35 is administered (see arrow D) is substantially perpendicular to the direction of flow C of the airflow so as to enable atomization of additive 35 to be optimized.

FIG. 6 shows a perspective view of a part of another system 43 according to the invention. FIG. 6 shows more particularly a passive, substantially plate-like atomizer body 44 manufactured in this exemplary embodiment from silicon. Atomizer body 44 is coupled to a respiratory tube 45 (or optionally a substantially T-shaped coupling element connected to respiratory tube 45), through which an airflow is guided in direction x. Atomizer body 44 is also connected to an administering conduit 46 provided with an additive. Atomizer body 44 comprises a plurality of atomizing channels 47 arranged in line and transversely of the airflow, which atomizing channels 47 form a passage for the additive present in administering conduit 46 to respiratory tube 45. Each atomizing channel has a length L of 0.2 millimetre and a diameter D of 2 micrometres. According to the formula

$$p = \frac{32 \cdot v \cdot η \cdot L}{{D}^{2}}$$

the operating pressure p which is exerted on the additive, at a velocity < of the additive of 20 m/s and with the use of an aqueous additive with a viscosity 0 of about 1, must be about 32 bar to enable atomizing of the additive as shown in this figure. The mutual distance y between atomizing channels 47 amounts in this exemplary embodiment to 20 micrometres.

FIG. 7 shows a schematic representation of an alternative embodiment of a system 47 for artificial respiration of a patient 48 according to the invention. System 47 comprises a respirator device 49 and a respiratory tube 50 coupled to respirator device 49, which respiratory tube 50 is arranged partially in patient 48. System 47 is also adapted to administer a plurality of medications 51a, 51b to patient 48. Medications 51a, 51b are preserved separately in a plurality of supply containers 52a, 52b, which supply containers 52a, 52b are each coupled via an administering tube 53a, 53b to an atomizer body 54 connected to respiratory tube 50 for the purpose of enabling atomizing of medications 51a, 51b in an airflow to be inhaled. Although atomizing of medications 51a, 51b outside the patient 48 is preferred to atomizing medications 51a, 51b inside the patient 48, as described at length in the foregoing, system 47 according to the invention is flexible such that it would however optionally be possible to atomize the medications 51a, 51b inside patient 48 as shown in this figure. Atomizer body 54 is provided with a plurality of atomizing channels (not further shown) for passage of medications 51a, 51b, wherein the diameter of each atomizing channel amounts to a maximum of 2.5 micrometres and the length of each atomizing channel amounts to 0.05 millimetre. In order to enable atomizing of medications 51a, 51b, a force Fₐ, F_{b} can be selectively exerted by pressure-generating elements (not shown) on medications 51a, 51b present in supply containers 52a, 52b, such as for instance an operating pressure of 10 bar, 48 bar respectively. These operating pressures are sufficiently high to enable reliable atomizing of the medication while being sufficiently low to enable a good sealing of system 47, whereby leakages in system 47 can be prevented. In the shown exemplary embodiment the pressure-generating elements and respirator device 49 are coupled to a control unit 55 for the purpose of enabling selective atomizing of at least one medication 51a, 51b subject to the situation of respirator device 49. Control unit 55, and optionally also supply containers 52a, 52b, can optionally be arranged integrally in respirator device 49.

Administering tubes 53a, 53b are connected to each other as a multi-lumen tube and are together guided in respiratory tube 17 close to patient 48. Because the part of respiratory tube 50 situated in patient 48 encloses administering tubes 53a, 53b, the assembly of tubes 50, 53a, 53b can be arranged relatively easily and reliably in patient 48.

It will be apparent that the invention is not limited to the exemplary embodiments shown and described here, but that numerous variants, which will be self-evident to the skilled person in this field, are possible within the scope of the appended claims.

1-43. (canceled)

44. A system for artificial respiration of persons, comprising:
a respirator apparatus, comprising:
a respirator device, and
at least one respiratory tube coupled to the respirator device, which
respiratory tube is adapted to enable respiration of a person; and
at least one atomizing device for atomizing an additive in an airflow to be inhaled by the person; comprising:
at least one passive, substantially plate-like atomizer body provided with at least one perforation defining an atomizing channel,
at least one supply holder for holding a supply of at least one substantially liquid additive,
at least one administering conduit for the additive connected to the supply container and the at least one atomizing channel of the atomizer body, and
pressure-generating means coupled to the supply container to enable a pressure to be exerted on the additive such that the additive can be atomized by means of the atomizer body into the airflow to be inhaled,
wherein the at least one atomizing channel of the passive atomizer body has a diameter of a maximum of 5 micrometres, and wherein the length of the administering conduit amounts to at least one metre.

45. The system as claimed in claim 44, wherein the atomizer body is coupled to the respirator apparatus such that atomizing of the additive in the airflow takes place at a distance from the person to be respirated.

46. The system as claimed in claim 45, wherein the atomizer body is coupled to the respirator apparatus such that atomizing of the additive in the airflow takes place at a distance of a maximum of 20 centimetres from the person to be respirated.

47. The system as claimed in claim 44, wherein the atomizer body is coupled to a half of the respiratory tube directed toward the person.

48. The system as claimed in claim 44, wherein a proximal outer end of the respiratory tube is provided with at least one intubation element adapted to be at least partially inserted into the person to be respirated.

49. The system as claimed in claim 48, wherein the atomizer body is adapted to atomize the additive in the airflow at a distance from the part of the intubation element inserted into the person to be respirated.

50. The system as claimed in claim 44, wherein a proximal outer end of the respiratory tube is provided with at least one respiratory cover adapted to fit onto the person to be respirated.

51. The system as claimed in claim 44, wherein the atomizer body is connected to a part of the respiratory tube located a distance from the intubation element, to a part of the intubation element not to be inserted into the person and/or to the respiratory cover.

52. The system as claimed in claim 44, wherein the diameter is substantially constant in the length direction of the at least one atomizing channel.

53. The system as claimed in claim 44, wherein the at least one atomizing channel of the atomizer body takes a substantially linear form.

54. The system as claimed in claim 44, wherein the atomizer body is provided with a plurality of perforations, wherein each perforation defines an atomizing channel, and wherein each atomizing channel has a diameter of a maximum of 5 micrometres.

55. The system as claimed in claim 54, wherein the atomizing channels have a substantially identical diameter.

56. The system as claimed in claim 54, wherein the mutual distance between the atomizing channels amounts to a maximum of 50 micrometres.

57. The system as claimed in claim 52, wherein the atomizing channels are arranged mutually in line.

58. The system as claimed in claim 57, wherein the atomizing channels are arranged mutually in line and substantially transversely of the airflow to be inhaled.

59. The system as claimed in claim 44, wherein the atomizer body is adapted to generate an atomized additive with an average droplet size of a maximum of 10 micrometres.

60. The system as claimed in claim 44, wherein the atomizer body is adapted to generate an atomized additive with a geometric standard deviation (GSD) lying between 1.001 and 1.2.

61. The system as claimed in claim 44, wherein the length of the at least one atomizing channel amounts to a maximum of 0.2 millimetre.

62. The system as claimed in claim 44, wherein the pressure-generating means are adapted to exert on the additive an operating pressure p substantially equal to
$$p = \frac{32 \cdot v \cdot η \cdot L}{{D}^{2}} ,$$
wherein < relates to the velocity of the additive, 0 to the viscosity of the additive, L to the length of the atomizing channel and D to the diameter of the atomizing channel.

63. The system as claimed in claim 44, wherein the pressure-generating means are adapted to exert on the additive an operating pressure of a maximum of 50 bar, in particular an operating pressure lying between 5 and 25 bar.

64. The system as claimed in claim 44, wherein the atomizer body is manufactured at least partially from at least one of the following materials: plastic, metal and ceramic.

65. The system as claimed in claim 44, wherein the diameter of the at least one atomizing opening lies substantially between 0.5 and 5 micrometres.

66. The system as claimed in claim 44, wherein the length of administering conduit is at least 150 cm and in particular 250 cm.

67. The system as claimed in claim 44, wherein the smallest diameter of the administering conduit lies substantially between 0.05 and 2 mm.

68. The system as claimed in claim 67, wherein the diameter of the administering conduit amounts substantially to 0.5 mm.

69. The system as claimed in claim 44, wherein the atomizing device comprises a plurality of supply containers.

70. The system as claimed in claim 69, wherein each supply container is coupled to its own administering conduit.

71. The system as claimed in claim 44, wherein the atomizing device comprises a plurality of atomizer bodies.

72. The system as claimed in claim 71, wherein the administering conduit connects to a plurality of atomizer bodies.

73. The system as claimed in claim 71, wherein the different administering conduits are coupled to different atomizer bodies.

74. The system as claimed in claim 73, wherein the different atomizer bodies are positioned adjacently of each other.

75. The system as claimed in claim 44, wherein the administering conduit is manufactured at least partly from a material substantially having bending stiffness.

76. The system as claimed in claim 44, wherein the atomizing device comprises at least one coupling element for coupling the atomizer body to the respiratory tube.

77. The system as claimed in claim 76, wherein the coupling element takes a substantially T-shaped form.

78. The system as claimed in claim 76, wherein an inner side of the coupling element defines a mixing zone for mixing the airflow with the atomized additive, wherein the area A of a cross-section of the mixing zone changes in the flow direction x of the airflow such that the change dA(x)dx at any location x in the mixing zone lies between −c₁√A(x) and 0 or between c₂√A(x) and 0, wherein c₁=15.35 or 4.22 and c₂=1.58 or 0.88 or 0.31.

79. The system as claimed in claim 44, wherein the atomizing device comprises at least one closing valve for regulating passage of the additive through the administering conduit.

80. The system as claimed in claim 79, wherein the system comprises at least one control unit for controlling the at least one closing valve subject to the situation of the respirator device.

81. The system as claimed in claim 80, wherein the respirator device comprises at least one flow sensor for detecting the flow in the respiratory tube, and that the control unit is coupled to the at least one flow sensor for the purpose of controlling the at least one closing valve.

82. The system as claimed in claim 44, wherein the administering conduit and the atomizer body take a disposable form.

83. The system as claimed in claim 44, wherein the atomizing device and the respirator apparatus are at least partly integrated with each other.

84. An atomizing device for use in a system as claimed in claim 44.

85. An assembly of an administering conduit and an atomizer body for use in an atomizing device as claimed in claim 84.

86. An atomizer body for use in an assembly as claimed in claim 85.
